(19) United States
(12) Patent Application Publication
(10) Pub. No.: US 2010/0211109 A1
(43) Pub. Date: Aug. 19, 2010

(54) TACK FOR SPINE FIXATION

(76) Inventor: TIMOTHY E. DOERR, BOISE, ID (US)

Correspondence Address: PEDERSEN & COMPANY, PLLC, P.O. BOX 2666, BOISE, ID, 83701, US

(21) Appl. No.: 12/541,912

(22) Filed: Aug. 14, 2009

Related U.S. Application Data
(60) Provisional application No. 61/088,793, filed on Aug. 14, 2008.
(60) Provisional application No. 61/097,095, filed on Sep. 15, 2008.
(60) Provisional application No. 61/161,074, filed on Mar. 18, 2009.

(51) Int. Cl. A61B 17/88; A61B 17/86

(52) U.S. Cl. 606/279; 606/329

(57) ABSTRACT

A tack for insertion into facets of the human spine is made of one or more bioactive materials. The tack is preferably pushed/impacted axially into holes in said facets, rather than rotated or screwed into said holes/facets. The tack may be installed at the posterior side of the lumbar region of the spine, to either fix facets of two vertebrae together or to fix facets of the lowermost vertebra to facets of the sacrum. The preferred tack may be made with barbs or other protrusions that resist or prevent the tack from backing out of the holes of the facets, or may be made without barbs and without protrusions, other than the natural surface texture or porous texture of the preferred porous metal(s) material. The barb-less and protrusion-less tacks are believed to be effective in remaining inside the holes/facets by virtue of their surface texture, which at least in part results from their porosity. Preferably, the tack is not threaded, and is installed with little, and preferably no, rotation of the tack on its longitudinal axis.

This application claims benefit of Provisional Application Ser. No. 61/088,793, filed Aug. 14, 2008, Provisional Application Ser. No. 61/097,095, filed Sep. 15, 2008, and Provisional Application Ser. No. 61/161,074, filed Mar. 18, 2009, the entire disclosures of which are incorporated herein by this reference.

### BACKGROUND OF THE INVENTION

1. Field of the Invention

The invention relates generally to apparatus for fixation of portions of the human spine, and, more particularly, to apparatus for fixing one vertebra to another, or one vertebra to the sacrum. The preferred apparatus is a tack, made from bioactive materials, that is axially inserted into holes in facets of said vertebra(e) and/or sacrum, rather than being screwed into said holes. Said preferred tack is surprisingly the only structure needed for posterior fixation of the spine, and the tack preferably does not connect to, and is not an anchor or fastener for, any supplemental fixation or support structure such as bars, brackets or plates. The preferred tack is used on posterior surfaces of the vertebra(e) and sacrum, preferably in combination with a fixation plate that is attached to anterior surfaces of said vertebra(e) and/or sacrum.

2. Related Art

Screws and/or plates and arms have been used on the spine to fix portions of the spine together and/or perform other repair. For example, see Vichard (U.S. Pat. No. 5,318,567); Puno, et al. (U.S. Pat. No. 5,360,431); Ray (U.S. Pat. No. 5,527,312); Cornwall, et al. (U.S. Pat. No. 6,485,518); Berry, et al. (U.S. Patent Application 2006/0276788 A1); Culbert, et al. (U.S. Patent Application 2007/0118132 A1); and Berg, et al. (2008/0015585). These systems tend to be complex and include screwing of threaded members into bone.

Still, the inventor believes that there is a need for an improved implant/apparatus for spine fixation that is simple in structure and minimally-invasive. The inventor believes that there is a need for an improved implant/apparatus that is made of bioactive materials that allow or encourage bone growth into the implant/apparatus, preferably resulting in bone growth all the way through the implant/apparatus and/or replacement of the material of the implant/apparatus by bone growth. The inventor believes that there is a need for an improved implant/apparatus that is minimally invasive but that is sufficiently strong and durable so that it may be forced into holes in the vertebra(e) and/or sacrum without breaking.

### SUMMARY OF INVENTION

The invention comprises a tack for insertion into facets of the human spine, wherein the tack is made of one or more bioactive materials. The tack is preferably pushed/impacted/tapped axially into holes in said facets, rather than rotated or screwed into said holes/facets. In preferred embodiments, the tack is installed at the posterior side of the lumbar region of the spine, to either fix facets of two vertebrae together or to fix the facets of the lowermost vertebra to facets of the sacrum.

Preferably, the tack is not threaded, and is installed with little, and preferably no, rotation of the tack on its longitudinal axis. In a first group of embodiments, the tack is made with barbs or other protrusions that resist or prevent the tack from backing out of the holes of the facets. In a second group of embodiments, the tack is made without barbs and without other protrusions, and the material of the tack and its interaction with the bone is sufficient to resist or prevent the tack from backing out of the holes of the facets.

It is important and surprising that the preferred embodiments of posterior spine fixation apparatus consist essentially of, and preferably consist only of, one more tacks inserted into facets of the facet joints, to extend across one or more facet joints of the spine to fix said facet joints. In other words, only said preferred tacks are used to make the facets of the selected facet joint(s) substantially or entirely immovable relative to each other, so that said the vertebra(e)/sacrum of the spine no longer bend/move relative to each other at said selected facet joint(s). The preferred embodiments of posterior spine fixation, therefore, do not include any additional structure implanted into the body, for example, no bars, no plates, no screws, or other structure extending between portions of the vertebra(e) and/or sacrum, or from vertebra to vertebra, or from vertebra to sacrum. The preferred apparatus is surprising effective and its simplicity results in extremely non-invasive apparatus and surgery methods.

### BRIEF DESCRIPTION OF THE DRAWINGS

It may be noted that, in reference to the figures and the tacks shown therein, the terms “top end” and “bottom end” are used for convenience, with the bottom end being the end that leads during insertion into the body, and the top end being opposite the bottom end, but it will be understood that this terminology is not necessarily consistent with the orientation of the tacks when in use in the human body.

FIG. 1 is a front (anterior) view of one embodiment of a fixation plate installed on the lowermost vertebra and the sacrum of a lumbar region of a human spine. The fixation plate is one embodiment of a fixation device for the anterior surfaces of vertebra(e) and/or sacrum that may be used with embodiments of the invented tack.

FIG. 2A is a rear (posterior) view of the lumbar region of FIG. 1, wherein one embodiment of the invented tack is shown inserted into the inferior facets of the lowermost vertebra and into the superior facets of the sacrum, to fix the lowermost-vertebra-sacrum facet joint.

FIG. 2B is a posterior view of two vertebrae, wherein two tacks according to embodiments of the invention are installed in (“across”) facet joints of said two vertebrae. The superior facets of the lower vertebra are fixed to the inferior facets of the upper vertebra, to prevent movement and stabilize the facets joints, and thus, to limit/prevent movement of the two vertebra to each other.

FIG. 2C is a left side view of the lower spine, showing use of tacks according to the preferred embodiments between the sacrum and the lowermost vertebra and also at three other locations between vertebrae. Also, anterior plates are shown in two locations, in dashed lines, to indicate that one or more plates may be used in combination with the posterior tacks. Preferably, said anterior plates screwed or fixed to the anterior surfaces of the spine are the only anterior fixation/fusion apparatus and the posterior tacks are the only posterior fixation/fusion apparatus. It should be noted that a surgeon will not necessarily install all of the tacks shown and/or all the plates shown, but may install tacks and/or plates in one or more of these locations, for example, as needed for the particular patient.

FIG. 2D is a left, partial view of a facet joint fixed by an embodiment of the invented tack.

FIG. 3 is a side view of one embodiment of the invented tack installed into a facet joint, specifically into an interior facet and a superior facet, wherein the bone facets are shown in cross-section.

FIG. 4 is a side view of another embodiment of the invented tack, wherein the protrusions are intended to abut/grip the wall surface of a hole drilled across the facet joint. The protrusions are slanted relative to the axial dimension of the tack, but said protrusions are not meant to be threads. The protrusions are merely for abutting/gripping the wall surface upon axial insertion of the tack into the hole, and the tack is preferably not to be rotated into the hole.

FIG. 5A is a side view of another embodiment of the invented tack, which has protrusions extending from left and right sides of the tack, but not extending from locations 360 degrees around the tack.

FIG. 5B is a side view of the embodiment of the invented tack shown in FIG. 3, which has protrusions extending from the tack axial side surface 360 degrees around the tack.

FIG. 6 is a side view of another embodiment of the invented tack, wherein the (bottom) tip of the tack is rounded, protrusions extend from the side surface of the tack near the tip and axial slots are provided near the top end of the tack.

FIG. 7 is a side view of another embodiment of the invented tack, wherein multiple, shelf-like protrusions extend from the tack axial side surface in four locations around the tack (for example, spaced generally 90 degrees).

FIG. 8 is a side view of another embodiment of the invented tack installed into a facet joint, specifically into an interior facet and a superior facet, wherein the bone facets are shown in cross-section. This tack is similar to that shown in FIGS. 3 and 5B, but comprises an enlarged top end.

FIG. 9 is a side view of another embodiment of the invented tack, prior to installation in the facets, which tack is similar to that in FIG. 4 except that this tack has an enlarged top end. As in the embodiment of FIG. 4, the protrusions are slanted relative to the axial dimension of the tack, but said protrusions are not meant to be threads. The protrusions are intended to abut/grip the wall surface of a hole drilled across the facet joint upon axial insertion of the tack into the hole, but the tack is preferably not to be rotated into the hole.

FIG. 10 is a side view of another embodiment of the invented tack, which is similar to the tack of FIGS. 3 and 5B, except that this tack has an enlarged top end. This tack has protrusions extending from locations 360 degrees around the tack for abutting/gripping the hole wall surface.

FIG. 11 is a side view of another embodiment of the invented tack that is similar to the tack in FIG. 6, except that this tack has a slightly enlarged top end, so that the outer diameter of the tack is smallest at the bottom end and is larger nearer the top end.

FIG. 12 is a side view of another embodiment of the invented tack, which is similar to the tack in FIG. 7 except that this tack has an enlarged top end.

FIG. 13 is a side view of another embodiment of the invented tack installed into a facet joint, specifically into an inferior facet and a superior facet, wherein the bone facets are shown in cross-section. This tack main body is cylindrical in shape with a rounded bottom end, and has with a smaller-diameter cylinder protruding upward from the main body of the tack to create a radial shelf, at the junction between the larger-diameter and smaller-diameter portions of the tack. The top end (also “proximal end”) that is smaller in diameter than the main body of the tack and said radial shelf may be used for engagement and/or impacting by the tool that is used to axially-force the tack into the facet hole. This tack comprises no barbs or protrusions formed or added to the side surface of the tack. Preferably, the tack is made from a porous metal(s), as discussed in more detail below, that has surface texture due to said porosity that grips the wall surface of the facet hole and, hence, tends to retain the tack in the facet hole.

FIG. 14 is a side view of another embodiment of the invented tack installed into a facet joint, specifically into an inferior facet and a superior facet, wherein the bone facets are shown in cross-section. This tack comprises no barbs or protrusions formed or added to the side surface of the tack, and has a proximal end (top end) that is larger in diameter than the main body of the tack. This tack main body is cylindrical in shape with a rounded bottom end. The top end (also “proximal end”) forms a slightly-enlarged (relative to the main body) radial shelf or radial surface that may be used for engagement and/or impacting by the tool that is used to axially-force the tack into the facet hole. Optionally (and not shown) an indentation may be provided in the top end of this tack, for example, at the axial centerline, to assist in said engagement/impacting by said tool. This tack comprises no barbs or protrusions formed or added to the side surface of the tack. Preferably, the tack is made from a porous metal(s), as discussed in more detail below, that has surface texture due to said porosity that grips the wall surface of the facet hole and, hence, tends to retain the tack in the facet hole.

FIGS. 15-17 schematically illustrate methods of installing embodiments of the invented tack in a spine, wherein FIG. 15 schematically illustrates a method of docking the trocar/sheath T on the medial facet of the joint to be stabilized; FIG. 16 schematically illustrates a step wherein a drill guide and drill may be inserted into the sheath the drill advanced through the midportion of the medial facet across the facet joint; and FIG. 17 schematically illustrates a tack guide/impact tool IT being placed through a sheath for impacting across the facet joint, for example, by a mallet MT. In FIG. 16, dashed lines are used to indicate that various trocar/sheath T apparatus and/or various insertion devices may be used as guide and control devices for the insertion and location, followed by impacting, of the tack into the facet hole, as will be understood after reading and viewing this disclosure.

FIG. 18A illustrates an example of a surface texture of one embodiment of a tack according to the invention, that is, the tack of FIG. 14, which is a rough/uneven texture resulting from porosity of the material from which the tack is made. At least the sidewall of the tack, 360 degrees around the tack, has this desirable texture, and, typically, all of the exterior surface of the tack has this desirable texture.

FIG. 18B illustrates a microscopic view of the preferred porous metal(s) of which the tack of FIG. 18A is made, which results in a texture that is adapted for excellent gripping of the facet hole wall surface and adapted for excellent bone growth into the porous structure of the tack after the tack has been installed.

### DETAILED DESCRIPTION OF THE PREFERRED EMBODIMENTS

Referring to the Figures, there are shown several, but not the only, embodiments of the invented tack for spine fixation and examples of uses of the tacks in spine operations. Tacks according to embodiments of the invention may be used in various locations along the lumbar region of the spine, for example.

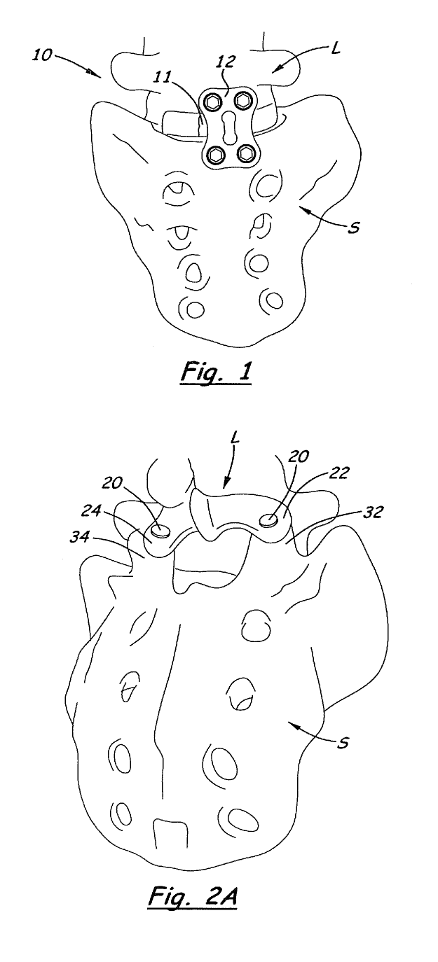

Preferably, the invented tacks are used in combination with an anterior fixation plate, such as that shown in FIG. 1. FIG. 1 is an anterior view of the lower lumbar region 10 of the spine, wherein one embodiment of an interbody implant 11 (preferably allograft), has been installed between the lowermost vertebra L and the sacrum S, and a fixation plate 12 has been installed on the anterior surfaces of said lowermost vertebra and sacrum. The rigid fixation plate 12 is screwed to said anterior surfaces, in order to fix that vertebra-sacrum joint and prevent relative movement of said vertebra and sacrum. One design of fixation plate 12, the Antegra™ device, may be obtained from Synthes, with U.S. offices in West Chester, Pa.

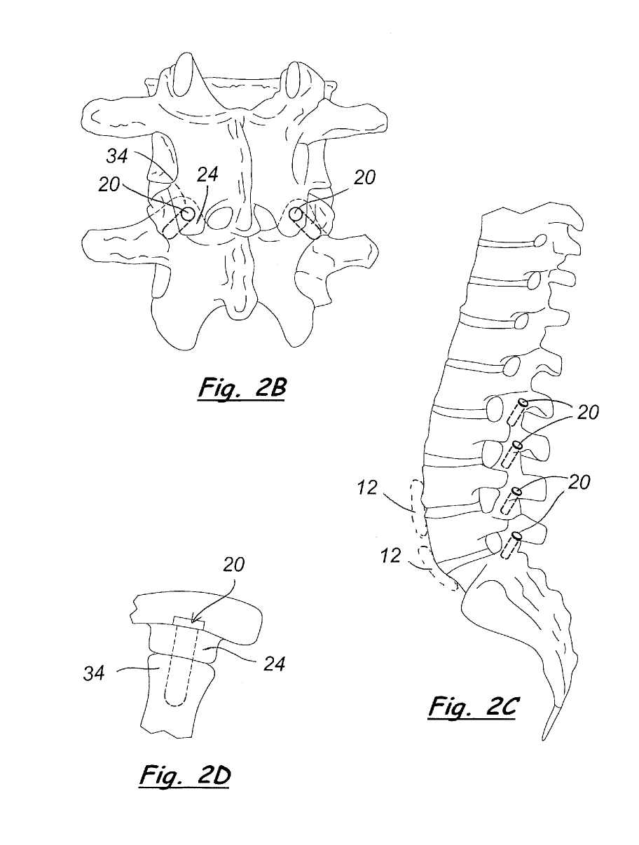

FIG. 2 illustrates the posterior region of the lumbar region 10, wherein an embodiment of the invented tack (tack 20) is used to fix inferior right and left facets 22, 24 of the lowermost vertebra L to the superior right and left facets 32, 34 of the sacrum S. Tacks 20 are installed in holes 41, 42 drilled into said facets 22, 24, 32, 34, preferably by pushing or punching the tacks 20 and not by rotating or screwing-in the tacks 20. Therefore, tacks 20 are adapted, and the methods established, for pushing into said holes 41, 42 and not screwing into said holes 41, 42. Preferably, the tacks are not rotated at all when being installed (0 degrees rotation), or, at most are rotated only slightly, for example, incidental/accidental rotation less than 10 degrees and more preferably less than 5 degrees.

It may be noted that there is preferably no nut, fastener, or cap for the distal end of the tack. When used to connect a first facet and a second facet, the preferred tack extends through the first facet and deep into the second facet and/or all the way through the second facet. Note that some of the Figures portray the facet hole extending all the way through the second facet and some of the Figures portray the facet hole extending part way through the second facet. There is preferably no nut, fastener, or cap that is threaded or otherwise attached on the distal end of the tack and no nut, fastener, or cap that is threaded or otherwise attached on the proximal end of the tack (except that the proximal end may be enlarged or otherwise formed for improved handling and insertion during surgery). Instead, the tack connects and “fixes” the first and second facet to each other by means of said extending through and into, and by gripping, the two facets facet but not by being fastened or “capped” at a distal end protruding out of the bone.

In addition, it is preferred that there is no other non-bone structure associated with, extending from, fastened by, the tacks, so that the preferred posterior fixation apparatus consists of (closed language) the tacks and no other elements. This simple apparatus, consisting only of two preferred tacks, represents an extremely non-invasive apparatus and surgical methods for fixing the spine. The preferred tacks are, therefore, not fasteners for anchoring other non-bone elements to or around the spine, but are themselves the fixation apparatus. One may therefore differentiate the preferred tacks from bone screws that fasten other elements to the spine, such as are mentioned in the Related Art section of this document. Thus, the terminology “a posterior spine fixation apparatus consisting of one or more tacks” or “a posterior spine fixation apparatus consisting of a plurality of tacks” or here and in the claims means that only the tack (preferably two tacks, a single tack in a right facet joint and a single tack in a left facet joint), with no bars, plates, extensions, supports, or other non-bone elements attached to, or extending from, the tack, is/are the posterior fixation apparatus.

Also, it is preferred that, when both anterior fixation and posterior fixation are used, that the anterior fixation is as simple and non-invasive as possible. For example, it is preferred that a simple plate, such as the plate shown in FIG. 1, is fixed to the anterior surface of the sacrum and the lowermost vertebra or to two adjacent vertebra, again with no bars, plates, extensions, supports, or other non-bone elements attached to, or extending from, the plate except that screws preferably secure the plate to said sacrum and vertebra. “Adjacent vertebrae” herein means one directly above the other in the spine. Therefore, the terminology “a spine fixation apparatus consisting of a plate fastened to anterior surfaces of two adjacent vertebrae and/or to anterior surfaces of a sacrum and an adjacent vertebra, and two tacks fixing posterior left and right facet joints . . . ” here and in the claims means that the anterior fixation device is only a plate screwed (or otherwise pinned, anchored, or fastened) to said vertebrae or said sacrum and a vertebra (and no other elements) and only two tacks, one in a right facet joint and one in a left facet joint), without additional bars, plates, extensions, supports, or other elements attached to, or extending from, the plate and the tacks. Therefore, in the preferred fixation apparatus, there are no elements extending between said plate and said tacks, no elements extending around any portion of the spine, and no elements protruding outward from the plate and/or the tacks to extend to other portions of the spine.

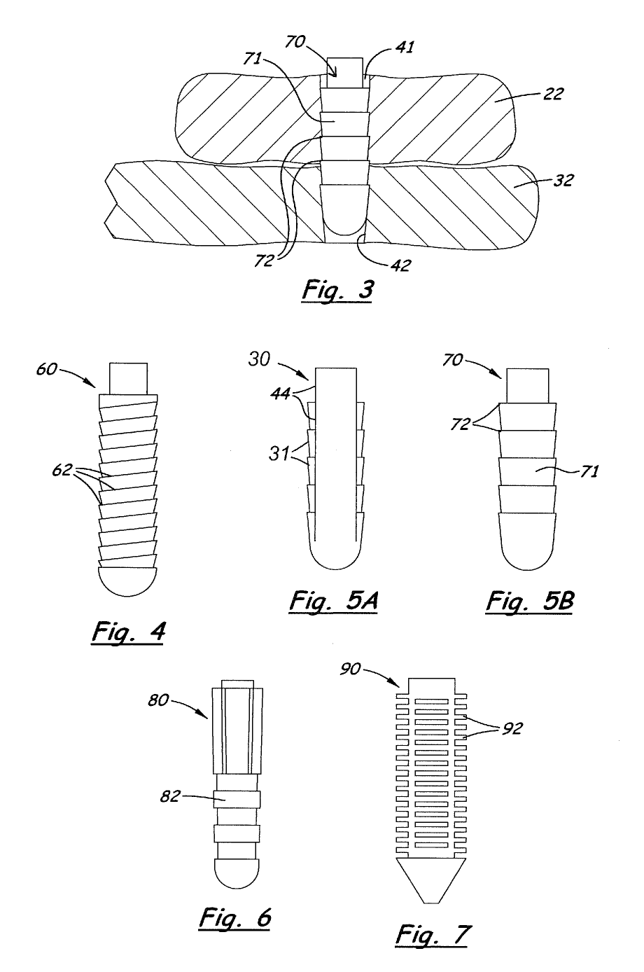

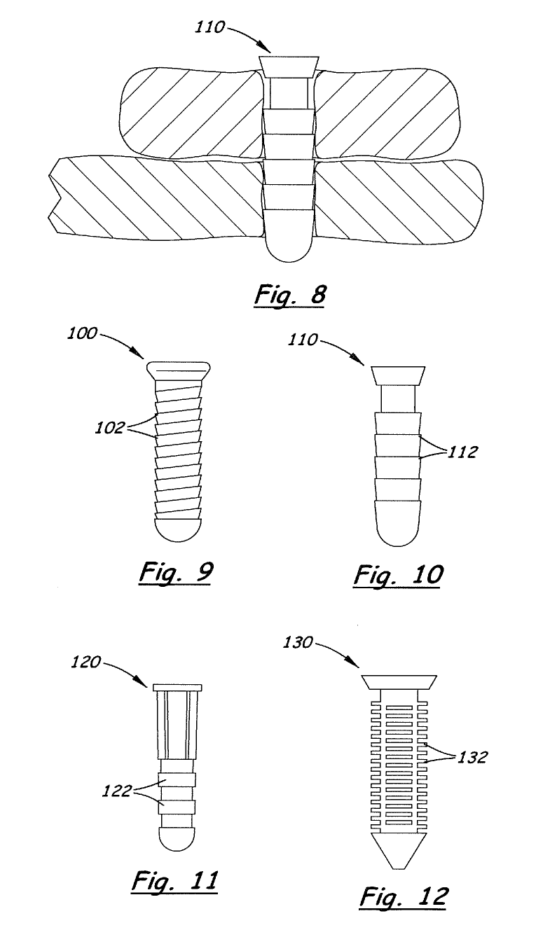

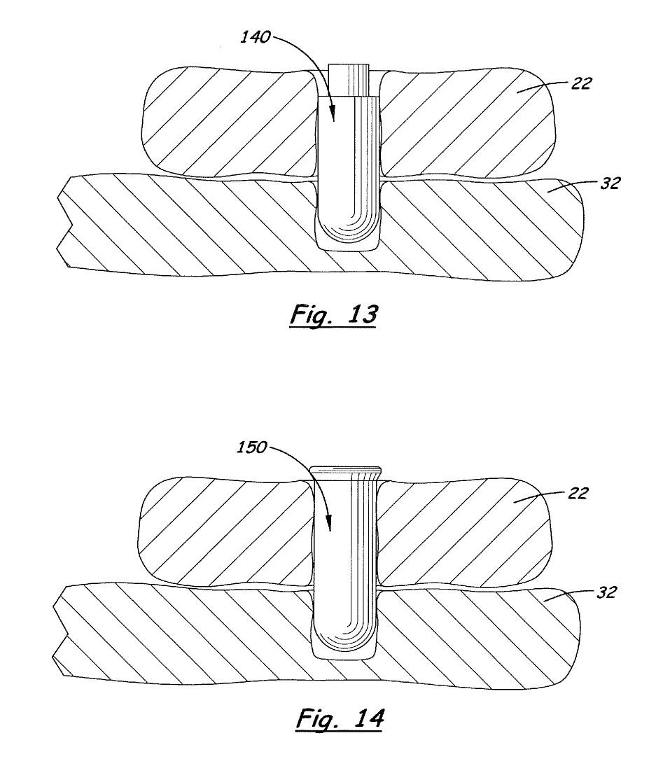

Also, while the proximal end of the tack may comprise an enlarged end or “tack head” in some embodiments (for example, see FIGS. 2 and 8-12), the proximal end of the tack may not be enlarged relative to the main body of the tack or may even be of smaller diameter than the main body of the tack (for example, see FIGS. 3-7). Thus, it is not necessarily required to have an enlarged proximal end, or other enlarged cap or fastener on the proximal end of the main body. The smaller diameter proximal end may be useful as a means for a hollow-ended tool to surround and capture said proximal end, so that the tack may be impacted (through the tool) without the tool sliding off of the tack and without the tool gouging or chipping the bone or adjacent soft tissue, or otherwise enlarging the hole in the bone during the impact. Alternatively, an enlarged-proximal-end tack, and/or the insertion or impact tool, may be adapted to cooperate during insertion and/or impact, to also allow capture or guiding of the proximal end and to prevent said sliding, gouging, or chipping. After the initial drilling of the hole, the tool(s) used to impact/install the tack preferably do not need to impact or touch the bone.

The tacks 20 are preferably made of bioactive material that promotes/accepts bone growth either by virtue of the bioactive material having pores that match or accept natural bone growth or by virtue of being made of material that is naturally replaced by growing bone, for example, in “resorption” or absorption” of the bioactive material and replacement of it by growing bone. The preferred tacks 20 are made from one or more of the following bioactive materials: machined allograft, beta-tricalcium phosphate polymer, and porous tantalum or other porous metal or metal composite materials.

The preferred materials are strong in the axial direction, and tacks made from these materials are expected by the inventor to withstand the impact/force of being pushed/impacted into said holes 41, 42, even when the tacks are made to be very small (for example, 4-6 mm in diameter). It is important that the tacks be made to be very small in order to fit into/through the facets 22, 24, 32, 34, which are small bone portions protruding out from the vertebrae and sacrum, as is well know in the medical arts.

Holes are drilled, and the tacks chosen, for a close fit, and preferably even tight fit (but not risking breakage of the tack or the bone), between the hole wall (bone surface) and the tack generally cylindrical side surface and/or it protrusions. The tacks, preferably, do not bend or deform a significant amount, when impacted/forced into the hole, except, for example, deformation of portions of the axial side surface of the tack main body and/or protrusions therefrom on the order of approximately 0.1-1, as further described below in order for a tight fit to be obtained.

It is preferred that the tacks have main body surfaces (axial sidewall diameter) of less than or equal to 6 mm, and, more preferably, in the range of 4-6 mm. For example, in especially-preferred embodiments, cylindrical hole of 4.5 mm diameter is drilled through an inferior facet and into a corresponding superior facet. Then, a 5 mm tack is installed in the hole, wherein the main body largest diameter is 5.0 mm but the protrusions may extend out to increase the diameter by 0.5 mm diameter, for a outermost outer diameter of about 5.5 mm. Thus, this slightly larger-than-the-hole tack diameter will typically represent the main body being slightly larger than the hole (5.0 mm diameter main body not counting the protrusions) and the diameter of the protrusions of the tack being even larger (5.5 mm diameter overall counting the protrusions), so that forcing of the tack into the hole may deform the tack protrusions and possibly even the main body slightly, and/or may deform the bone hole surface slightly so that the tack becomes tightly installed in the hole and unlikely to “back out” of the hole. In the event that the hole has not been formed accurately-enough during drilling by the surgeon, and/or the tack or bone deforms too much to create a tight fit, a “salvage tack” of a larger diameter may be used, for example, a 5.5 mm diameter tack (5.5 mm at its largest diameter not counting the protrusions, for a total of approximately 6 mm with protrusions) to be installed in and securely remain in the inaccurate, nominal 4.5 mm hole.

In embodiments with no protrusions, such as are represented in FIGS. 2D, 13, and 14, the cylindrical diameter of the tack is preferably close to the diameter of the facet hole. For example, a 4.5 mm hole is drilled and the tack has an outer diameter (preferably consistent or nearly consistent all along the length of its main body) of 4.5-5.0, with a “salvage tack” having a diameter of 5.0-5.5 mm, for example. Depending on the accuracy of the hole formation and the precision of the tack diameter, a closer fit (4.6-4.8 mm for the tack to be secured held in a 4.5 mm hole) may be possible. It may be understood that various tack diameters may be effective for various hole sizes and drilling procedures, especially in view of the materials of manufacture of the tacks, and this will be determinable by one of skill in the art without undue experimentation.

The preferred bioactive materials are expected to be relatively brittle upon torsion, and, especially brittle when the tack is made to be very small (4-6 mm). The preferred tacks, therefore, are intended to be pushed or axially-impacted only, and not rotated or otherwise subjected to torsion. Therefore, while barbs or other protrusions may be provided on the outer axial surfaces of a first group of tack embodiments, it is preferred that these barbs/protrusions are not adapted to encourage or cause rotation of the tack in the holes and it is preferred that these barbs/protrusions are strong enough so that they do not snap or otherwise break when being installed, even if there is said incidental/accidental rotation. Therefore, while protrusions, such as the slanted protrusions in FIGS. 4 and 9, may be acceptable in some embodiments, it is preferred that such slanted protrusions are not threads that extend continuously around the main shaft of the tack and/or that the tack is purposely not rotated during installation and so no attempt is made to screw-in the tack.

The preferred posterior fixation system consists only of two tacks according to embodiments of the invention, and no additional bars, plates, arms, or hooks attached to the preferred tacks or on the posterior side of the lumbar region. Thus, preferably 90 percent or more of the apparatus for anterior fixation is installed inside/within the bone and does not protrude or lie along outer bone surfaces. Thus, the simplicity of the preferred apparatus and the minimally-invasive methods of installing the preferred apparatus provide benefits during and after surgery.

Each of the illustrated tacks 20, 30, 60, 70, 80, 90, 100, 110, 120, 130, 140, 150 is an elongated member, which may also be called a non-threaded pin or anchor because the tacks have no threads. Each of the tacks has a main body surface that is generally cylindrical along its entire length or, at least generally cylindrical along the portion of the tack that extends into the facets.

Extending from said main body surface of the example tacks of the first group of embodiments (tacks 30, 60, 70, 80, 90, 100, 110, 120, 130) are protrusions 31, 62, 62, 72, 82, 92, 112, 122, 132, some or all of which are preferably directed toward the top end so that they do not significantly interfere with insertion of the tack into the holes, and so that they do interfere with the tack backing up out of the holes during and after surgery.

In the second group of embodiments, the main bodies of tacks 20, 140 and 150, however, are generally cylindrical and do not have protrusions. The main bodies of tacks 20, 140 and 150 have sufficient texture, preferably of very small scale such as a rough and/or porous surface, that the main body tends to grip the bone surface of the hole into which it is impacted and resist or prevent the tack from backing up and out of the hole. In the case of the preferred bioactive materials, even a small amount of bone growth into the bioactive material will further increase the interaction and stability of the tack in the bone, so that the tack, over time, becomes even less likely to reverse itself out of the hole/facet. The preferred barb-less and protrusion-less tacks 20, 140, 150 do not have internal spaces or hollow regions other than the void space caused by the porosity of the material of the tack. Barb-less and protrusion-less tacks may have a recess (not shown) or other engagement structure on its proximal end that may cooperate with a trocar or other sheath, or other placement or impact tool, during installation of the tacks in the human body.

The preferred material for tack 140 and 150, and other protrusion-less or barb-less tacks, is Zimmer Trabecular Metal™ (see Zimmer.com and/or U.S. Pat. No. 5,282,861, which patent is hereby incorporated in its entirely into this disclosure by this reference), which is reported to be elemental tantalum metal material formed by vapor deposition techniques that create a metallic strut configuration similar to trabecular bone. Alternatively, other porous tantalum, porous tantalum-containing, or other porous metal(s) or porous-metal(s)-containing composite or alloys may be used that have a surface texture/porosity that grips the bone, without barbs and protrusions formed in, or attached to, the external side surface of the tack, and preferably encourages bone growth into and through the pores of the material.

In installation and use, one may see in the figures the preferred locations and methods of installation. The lumbar region of the spine is the particularly-preferred, but not necessarily the only, location for use of the invented tacks. Many patients with discogenic back pain have canal pathology (e.g., stenosis, migrated HNP) that requires posterior decompression, which has in the past prompted, and continues to prompt, many surgeons to choose an all-posterior approach with TLIF/PLIF and pedicle screw instrumentation which results in a large incision, more blood loss, paraspinal muscle denervation and increased risk of “fusion disease”. With the development of modern, stand-alone anterior lumbar plates (such as plate 12 in FIG. 1), minimally invasive anterior lumbar interbody fusions have become an attractive treatment option for the treatment of discogenic back pain. However, many surgeons have concerns with stand-alone anterior constructs (anterior constructs as the sole, only interbody fusion apparatus and methods), especially in the setting of previous or concurrent midline decompression with loss of the posterior ligamentous tension band. Therefore, the inventor believes that his facet fixation tack device designed specifically for lumbar facet immobilization and fusion will perform extremely effectively in combination with an anterior fusion apparatus.

Therefore, an especially-preferred combination for the lower spine, as portrayed to best advantage in FIGS. 1 and 2A, is an anterior fixation plate, an interbody allograft insert (as will be understood by those of skill in the art, given this disclosure), and two tacks according to embodiments of the invention, wherein the two tacks serve to fix the lowermost vertebra right and left facets to the sacrum superior right and left facts, respectively. Thus, the especially-preferred device provides immediate immobilization of the facet joint in order to augment anterior plating, and is biologically active and facilitates fusion of the facet joint. Thus, the preferred tacks are designed to be utilized as a minimally invasive adjunct to anterior lumbar interbody fusion procedures, and provide unique immobilization of facet joints with bioactive materials that preferably eventually become incorporated into a durable facet fusion.

The preferred tacks comprise the additional benefit and feature of being easily inserted through a posterior “microdiscectomy incision”. Thus, the especially-preferred embodiments are a fixation and fusion device designed to be utilized as a minimally-invasive adjunct to anterior lumbar interbody fusion procedures. The especially-preferred lumbar facet fixation device is designed to provide initial immobilization of the facet joint with bioactive materials that eventually become incorporated into a durable facet fusion. In many barbed embodiments, the cylindrical tack device will have an “inner” diameter (main body exterior diameter, not counting barbs/protrusions) of 5.0 mm, and a tapered or rounded leading surface to facilitate insertion. Barbs provided around the shaft (main body) may create an outer diameter (outermost diameter, counting barbs/protrusions) of 5.5 mm, for example. The lengths of the tacks are expected to range from 10 mm to 30 mm in 2 mm increments, for example. The “salvage” tacks, as discussed above, are expected to be approximately 5.5 mm inner diameter (not counting barbs/protrusions) and 6.0 mm outer diameter (counting barbs/protrusions). While various materials may be considered in the future, the current preferred materials for the preferred embodiments are machined allograft (preferably bone allograft), beta-tricalcium phosphate polymer, and/or porous tantalum or other porous metals/metal composites.

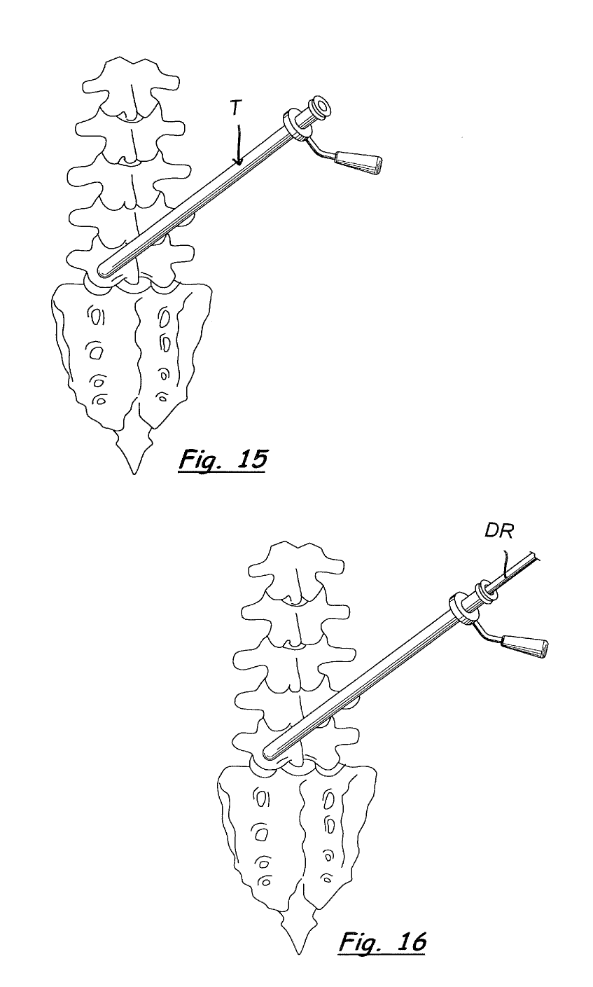

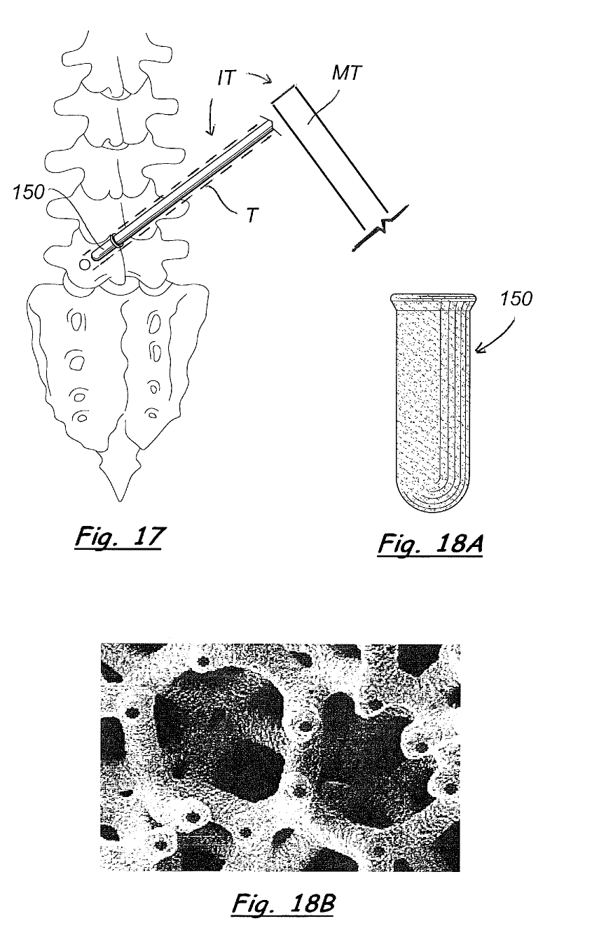

As may be seen schematically in FIGS. 15-17, the preferred methods of installation comprises a bilateral mini-laminotomy incision (not shown but understood in view of the figures by one of skill in the art), followed by a sheath/trocar T placed percutaneously and docked on medial facet. A 5.0 mm diameter drill DR may be advanced through midportion of the medial facet across the facet joint, exiting the lateral facet with a trajectory of dorsosuperomedial to anterioinferolateral. A tack of appropriate length is selected and may be inserted part way into the resulting hole in the inferior facet (herein called the “facet hole” or “holes”), due to the preferred tapered/rounded leading end of the tack, and then impacted across the facet joint, for example, by various placement and impacting tools IT, including a mallet MT or other means of providing force. One of skill in the art, upon reading and viewing this document and the Figures, will understand that various styles of preferably-non-invasive tools may be used for the installation steps or drilling, installing and impacting, to create a tight fit between the tack and the bone, wherein, as discussed above, the preferred installation steps do not comprise rotation of the tack upon its longitudinal axis. Once one of the preferred pair of tacks is installed, the steps are repeated across the contralateral facet joint.

A specific example of insertion techniques for the especially-preferred embodiments is as follows. In a minimally-invasive approach, if a midline decompression is required, a mini-laminotomy-incision is made in the midline exposing the posterior elements to the area of the medial facet joints bilaterally. A percutaneous stab incision is made superior and contralateral to the facet joint to be fixed with the tack. A sheath and trocar are then inserted through the stab incision between the interspinous ligaments or dorsal to the spinous processes. The sheath is docked on the medial facet of the joint to be stabilized. A drill guide is inserted into the sheath and a 5 millimeter drill is advanced through the midportion of the medial facet across the facet joint exiting the lateral facet with a projectory from dorsosupermedial to aneroinferolateral. The length of the tack required for the particular patient and the particular facet joint may be read directly off the drill guide, or alternatively may be measured with a depth gauge placed through the sheath. A tack guide is then placed through the sheath and an appropriate length tack is placed through the guide and impacted across the facet joint. This is then repeated in an identical fashion across the contralateral facet joint. It is recommended that the tack be placed prior to any lateral decompression to minimize the risk of facet fracture. If a midline decompression is not required, this technique can be utilized through two small muscle splitting incisions placed directly over the involved facet joints using the same percutaneous drill and tack technique. Alternative methods, and/or incision locations and projectories, may be used, depending on the patient, the patient's particular injury or spine damage, whether laminectomy is needed, and/or other issues; these issues will be understood by those in skill in the art and may be addressed without undue experimentation.

Other embodiments of the invented apparatus and methods will be apparent to one of skill in the art after reading this disclosure and viewing the drawings. Although this invention is described herein with reference to particular means, materials and embodiments, it is to be understood that the invention is not limited to these disclosed particulars, but extends instead to all equivalents within the broad scope of the following claims.

1. A posterior spine fixation apparatus consisting of a plurality of tacks, wherein each of said tacks is made from material selected from bone allograft, beta-tricalcium phosphate polymer, porous tantalum, porous metal, and porous metal composites.

2. The posterior spine fixation apparatus of claim 1, wherein each of said tacks is generally cylindrical and has a diameter in the range of 4-6 mm, and a length in the range of to range from 10 mm to 30 mm adapted for insertion through an inferior medial facet of a spine vertebra and into a superior facet of a sacrum.

3. The posterior spine fixation apparatus of claim 1, wherein each of said tacks is generally cylindrical and has a diameter in the range of 4-6 mm, and a length in the range of to range from 10 mm to 30 mm adapted for insertion through an inferior facet of a first spine vertebra and into a superior facet of a spine second vertebra.

4. The posterior spine fixation apparatus of claim 1, wherein each of said tacks has a generally cylindrical main body and has no protrusions extending radially from said main body, and wherein each of said tacks is made from material selected from the group of porous tantalum, porous metal, and porous metal composites so that the main body has an axial side surface that comprises porosity and has a texture formed by said porosity, said texture being adapted to grip a bone surface of a hole in an inferior facet and a bone surface of a hole in a superior facet of the spine.

5. The posterior spine fixation apparatus of claim 1, wherein each of said tacks has a generally cylindrical main body and has multiple protrusions extending radially from said main body, and wherein each tack is not threaded and is adapted to be inserted into a hole in an inferior facet and into a hole in a superior facet of the spine by axial pushing of the tack and not by rotation of the tack on the longitudinal axis of the tack.

6. A posterior spine fixation apparatus consisting only of a plurality of tacks, a single tack of said plurality being adapted for insertion across a right facet joint of a lower spine, and a single tack of said plurality being adapted for insertion across a left facet joint of the lower spine, wherein said posterior spine fixation apparatus comprises no bars, no plates, no extensions, and no supports attached to, or extending from, the tack.

7. The posterior spine fixation apparatus of claim 6, wherein each of said tacks is generally cylindrical and has a diameter in the range of 4-6 mm, and a length in the range of to range from 10 mm to 30 mm adapted for insertion through an inferior facet and into a superior facet of facet joint of a spine.

8. The posterior spine fixation apparatus of claim 6, wherein each of said tacks has a generally cylindrical main body and has no protrusions extending radially from said main body, and wherein each of said tacks is made from material selected from the group of porous tantalum, porous metal, and porous metal composites so that the main body has an axial side surface that comprises porosity and has a texture formed by said porosity, said texture being adapted to grip a bone surface of said inferior facet and a bone surface of said superior facet.

9. The posterior spine fixation apparatus of claim 6, wherein each of said tacks has a generally cylindrical main body and has multiple protrusions extending radially from said main body, and wherein each tack is not threaded and is adapted to be inserted into a hole in the inferior facet and into a hole in the superior facet of the spine by axial pushing of the tack and not by rotation of the tack on the longitudinal axis of the tack.

10. A spine fixation apparatus consisting only of a plate fastened to anterior surfaces of two adjacent vertebrae by a plurality of screws, and two tacks fixing posterior left and right facet joints of said two adjacent vertebrae.

11. A spine fixation apparatus consisting only of a plate fastened to anterior surfaces of a sacrum and an adjacent vertebra by a plurality of screws, and two tacks fixing posterior left and right facet joints of said sacrum and adjacent vertebra.

12. A method of facet fixation and fusion for a spine, the method comprising:
providing a plurality of tacks for posterior fixation of two adjacent vertebrae, wherein each of said tacks is made from material selected from bone allograft, beta-tricalcium phosphate polymer, porous tantalum, porous metal, and porous metal composites;
drilling through a right facet joint of two adjacent vertebrae to form a right hole through an inferior facet of the right facet joint and into a superior facet of the right facet joint;
drilling through a left facet joint of two adjacent vertebrae to form a left hole through an inferior facet of the left facet joint and into a superior facet of the left facet joint;
impacting one of said tacks into the right hole and impacting one of said tacks into the left hole, wherein only said tacks are used for fixation and fusing of posterior surfaces of said two adjacent vertebrae no other elements are attached to posterior surfaces of said two adjacent vertebrae.

13. A method of facet fixation and fusion as in claim 12, further comprising:
providing a plate for anterior fixation, and screwing the plate to anterior surfaces of said two adjacent vertebrae, and wherein only said plate screwed to said anterior surfaces is used for fixation and fusing of anterior surfaces of said two adjacent vertebrae.

14. A method of facet fixation and fusion for a spine, the method comprising:
providing a plurality of tacks for posterior fixation of a sacrum and an adjacent vertebra, wherein each of said tacks is made from material selected from bone allograft, beta-tricalcium phosphate polymer, porous tantalum, porous metal, and porous metal composites;
drilling through a right facet joint of the sacrum and vertebra to form a right hole through an inferior facet of the right facet joint and into a superior facet of the right facet joint;
drilling through a left facet joint of the sacrum and vertebra to form a left hole through an inferior facet of the left facet joint and into a superior facet of the left facet joint;
impacting one of said tacks into the right hole and impacting one of said tacks into the left hole, wherein only said tacks are used for fixation and fusing of posterior surfaces of said sacrum and vertebra no other elements are attached to posterior surfaces of said sacrum and vertebra.

15. A method of facet fixation and fusion as in claim 12, further comprising:
providing a plate for anterior fixation, and screwing the plate to anterior surfaces of said sacrum and vertebra, and wherein only said plate screwed to said anterior surfaces is used for fixation and fusing of anterior surfaces of said sacrum and vertebra.

16. A method as in claim 12, wherein each of said tacks is barbless and grips the bone surfaces of the right hole and the left hole by means of each tack having an axial side surface having a porous texture.

17. A method as in claim 14, wherein each of said tacks is barbless and grips the bone surfaces of the right hole and the left hole by means of each tack having an axial side surface having a porous texture.

18. A method of facet fixation and fusion for a spine, the method comprising:
providing posterior fixation apparatus consisting only of two tacks for posterior fixation of two adjacent vertebrae, wherein each of said two tacks is made from material selected from bone allograft, beta-tricalcium phosphate polymer, porous tantalum, porous metal, and porous metal composites;
drilling through a right facet joint of two adjacent vertebrae to form a right hole through an inferior facet of the right facet joint and into a superior facet of the right facet joint;
drilling through a left facet joint of two adjacent vertebrae to form a left hole through an inferior facet of the left facet joint and into a superior facet of the left facet joint;
impacting one of said two tacks into the right hole and impacting the other of said two tacks into the left hole.

19. A method of facet fixation and fusion as in claim 18, further comprising:
providing anterior fixation apparatus consisting only of a plate fastened to anterior surfaces two adjacent vertebra.

20. The method of claim 18, wherein each of said two tacks consists of a main body with a rounded or tapered distal end for easing insertion into said right hole and said left hole, and a proximal end comprising a flat radial surface for cooperating with an impact tool during said impacting.

21. The method of claim 18, wherein each of said two tacks consists of a main body with a rounded or tapered distal end for easing insertion into said right hole and said left hole, and a proximal end comprising a flat radial surface for cooperating with an impact tool during said impacting, and protrusions extending generally radially outward from axial side surfaces of the main body for gripping bone surface of said right hole and said left hole.

22. A method of facet fixation and fusion for a spine, the method comprising:
providing posterior fixation apparatus consisting only of two tacks for posterior fixation of a sacrum and an adjacent vertebrae, wherein each of said two tacks is made from material selected from bone allograft, beta-tricalcium phosphate polymer, porous tantalum, porous metal, and porous metal composites;
drilling through a right facet joint of said sacrum and vertebra to form a right hole through an inferior facet of the right facet joint and into a superior facet of the right facet joint;
drilling through a left facet joint of said sacrum and vertebra to form a left hole through an inferior facet of the left facet joint and into a superior facet of the left facet joint;
impacting one of said two tacks into the right hole and impacting the other of said two tacks into the left hole.

23. A method of facet fixation and fusion as in claim 22, further comprising:
providing anterior fixation apparatus consisting only of a plate fastened to anterior surfaces said sacrum and vertebra.

24. The method of claim 22, wherein each of said two tacks consists of a main body with a rounded or tapered distal end for easing insertion into said right hole and said left hole, and a proximal end comprising a flat radial surface for cooperating with an impact tool during said impacting.

25. The method of claim 22, wherein each of said two tacks consists of a main body with a rounded or tapered distal end for easing insertion into said right hole and said left hole, and a proximal end comprising a flat radial surface for cooperating with an impact tool during said impacting, and protrusions extending generally radially outward from axial side surfaces of the main body for gripping bone surface of said right hole and said left hole.
